(19) United States
(12) Patent Application Publication
(10) Pub. No.: US 2016/0058961 A1
(43) Pub. Date: Mar. 3, 2016

(54) System and Method of Nasal Aromatic Delivery

(71) Applicant: Michael Haas, Mesa, AZ (US)

(72) Inventor: Michael Haas, Mesa, AZ (US)

(21) Appl. No.: 14/835,952

(22) Filed: Aug. 26, 2015

Related U.S. Application Data
(63) Continuation-in-part of application No. 14/473,782, filed on Aug. 29, 2014.

(51) Int. Cl. A61M 15/08

(52) U.S. Cl. CPC A61M 15/08; A61M 15/085

(57) ABSTRACT

A system and method are disclosed for administration of pharmacologically- or therapeutically-active essential oils by inhalation from a nose-attached device.

### CROSS-REFERENCE TO RELATED APPLICATIONS

This application is a continuation-in-part of U.S. patent application Ser. No. 14/473,782, filed on Aug. 29, 2014, entitled “System and Method of Nasal Aromatic Delivery”. U.S. patent application Ser. No. 14/473,782 is owned to the owner of the present application. The subject matter disclosed in U.S. patent application Ser. No. 14/473,782 is hereby incorporated by reference into the present disclosure as if fully set forth herein.

### TECHNICAL FIELD

The disclosure relates generally to a system and method of nasal aromatic compound delivery and specifically to a system for administration of therapeutic or pharmacologically beneficial essential oils by inhalation.

### BACKGROUND

Essential oils are known to have some therapeutic and pharmacological properties. Aromatherapy, for example, requires inhaling essential oil vapors, which are volatilized into the ambient air by heating a small amount of oil over a heat source, such as a candle. However, this has several drawbacks. First, because the oil disperses into the ambient air, the vapor is diffuse and lacks sufficient concentration for effective administration. Second, a candle is not convenient to use in most situations outside the home, and even if it were, all other persons present in the area would be forced to inhale the vapors from the oil. Last, heating essential oil has been shown to cause carcinogenic compounds to form from the oil or, at the very least, causes the oil to break down and lose some or all of its therapeutic or pharmacological properties. These drawbacks are undesirable.

### SUMMARY

A nasal aromatic delivery system is disclosed. The system includes a body with a reservoir and a plurality of reservoir passages, a first of the plurality of reservoir passages is fluidly coupled with a first restriction channel, a first accumulation chamber, and a first orifice, and a second of the plurality of reservoir passages is fluidly coupled with a second restriction channel, a second accumulation chamber, and a second orifice. The system further includes a plurality of arms coupled with the body, each of the plurality of arms coupled with a clip, wherein the clip is configured to couple to an inner surface of a nostril.

A nasal aromatic delivery system with insert is disclosed. The system includes a body with a reservoir and a plurality of reservoir passages. A first of the plurality of reservoir passages is fluidly coupled with a first odorant gap and a first accumulation chamber and a second of the plurality of reservoir passages is fluidly coupled with a second odorant gap and a second accumulation chamber. The system further includes a first insert removably coupled with the first accumulation chamber and including a first insert restriction channel and a second insert removably coupled with the second accumulation chamber and comprising a second insert restriction channel. The first insert restriction channel fluidly couples a first orifice to the reservoir and the second insert restriction channel fluidly couples a second orifice to the reservoir.

### BRIEF DESCRIPTION OF THE DRAWINGS

A more complete understanding of the present invention may be derived by referring to the detailed description when considered in connection with the following illustrative figures. In the figures, like reference numbers refer to like elements or acts throughout the figures.

FIG. 1 illustrates a nasal aromatic oil delivery system according to a preferred embodiment;

FIG. 2 illustrates the nasal aromatic delivery system of FIG. 1 in greater detail in accordance with the preferred embodiment;

FIGS. 3A-3I illustrate various embodiments of a nasal aromatic delivery system according to an embodiment;

FIGS. 4A-4B illustrate an external view of a nasal aromatic delivery system according to an embodiment;

FIG. 5 illustrates an alternate embodiment of a nasal aromatic oil delivery system according to an embodiment;

FIG. 6 illustrates an embodiment of a nasal aromatic oil delivery system comprising an interchangeable insert;

FIG. 7 illustrates an embodiment of a nasal aromatic delivery system according to a sport personal diffuser embodiment;

FIG. 8 illustrates an embodiment of wicks;

FIG. 9 illustrates a top view of the sport personal diffuser in accordance with an embodiment;

FIG. 10 illustrates a side view of the sport personal diffuser in accordance with an embodiment;

FIG. 11 illustrates the sport personal diffuser in greater detail in accordance with an embodiment;

FIG. 12 illustrates an embodiment of a nasal aromatic delivery system according to a medical personal diffuser embodiment.

### DETAILED DESCRIPTION

Systems and methods of nasal aromatic delivery system and applications of the invention presented herein are described below in the drawings and detailed description of the invention. Unless specifically noted, it is intended that the words and phrases herein be given their plain, ordinary, and accustomed meaning to those of ordinary skill in the applicable arts.

In the following description, and for the purposes of explanation, numerous specific details are set forth in order to provide a thorough understanding of the various aspects of the invention. It will be understood, however, by those skilled in the relevant arts, that embodiments of the present invention may be practiced without these specific details. In other instances, known structures and devices are shown and/or discussed more generally in order to avoid obscuring the invention. In many cases, a description of the operation is sufficient to enable one of ordinary skill in the applicable art to implement the various forms of the invention. It should be appreciated that there are many different and alternative configurations, devices and technologies to which the disclosed inventions may be applied. The full scope of the present disclosure is not limited to the examples described below.

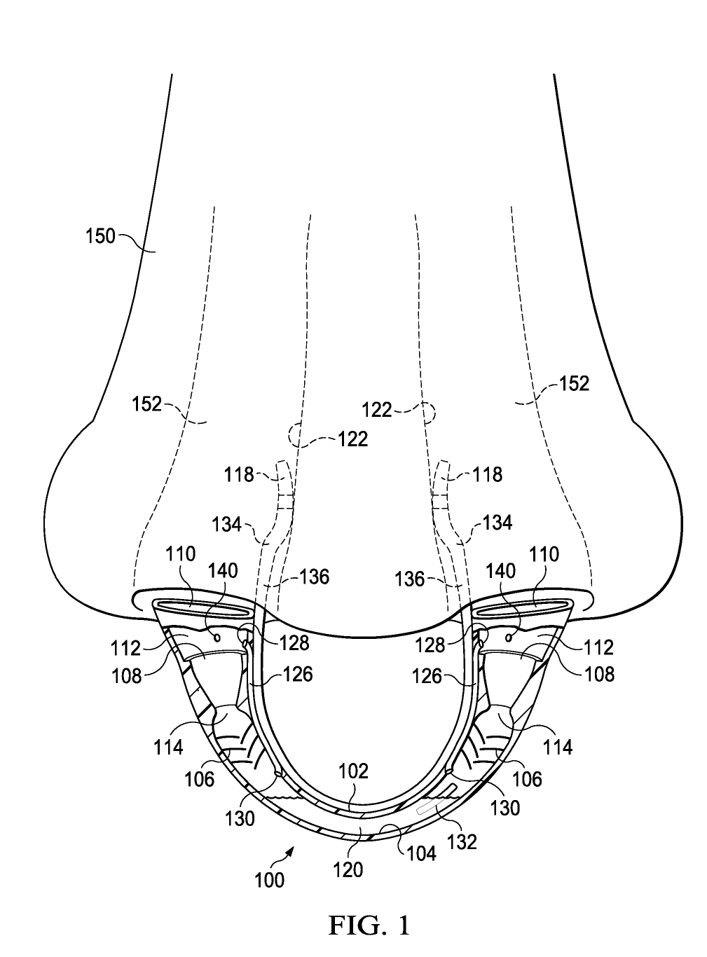

FIG. 1 illustrates a nasal aromatic oil delivery system 100 according to a preferred embodiment. Nasal aromatic delivery system 100 comprises one or more of the following: body 102, reservoir 104, louvers 106, fill port 108, orifice 110, accumulator chamber 112, restrictor channel 114, clip 118, pad attachment hole 124, channel 126, outlet 128, inlet 130, arm 136, and/or intake port 140.

Body 102 of nasal aromatic oil delivery system 100 comprises a durable and/or flexible material such as plastic, latex, silicon, rubber, or the like. In some embodiments, body 102 is transparent, translucent, opaque or a combination thereof. For example, body 102 comprises one or more windows 132 that allow the amount of odorant 120 inside reservoir 104 to be viewed from the outside. Alternatively, body 102 of nasal aromatic oil delivery system 100 may be entirely translucent or transparent to view the amount of odorant 120 inside reservoir 104 without one or more windows 132.

Body 102 is coupled with one or more clips 118 by one or more hinges 134 and/or arms 136. In some embodiments, body 102, clips 118, hinges 134 and arms 136 comprise a single unit and/or are constructed of a single material. According to some embodiments, one or more of body 102, clips 118, hinges 134, and/or arms 136 are constructed of one or more units and/or one or more materials such that hinges 134 provide a flexion point for clips 118 to secure tightly to inner surface 122 of nostril 152. For example, body 102, clips 118, hinges 134, and arms 136 may comprise a single piece of flexible plastic such that each clip 118 slides inside each nostril 152, and each clip 118 secures tightly to the inner surface 122 of nostril 152, with the force of the flexion of the one or more hinges 134 providing the force to hold nasal aromatic oil delivery system 100 securely inside of nose 150. Arms 136 couple body 102 to hinges 134. According to some embodiments, body 102 comprises the flexion point for nasal aromatic oil delivery system 100. For example, in some embodiments, body 102 comprises a flexible material, such as rubber, plastic, or the like, which bends sufficiently to provide a force against an inner surface 122 of a nostril 152 to hold clips 118 securely to the inside of nose 150.

A further feature of this attachment is that the force of the flexion of nasal aromatic oil delivery system 100 or hinges 134 dilates the nostrils 152 to provide increased airflow through nose 150, permitting easier breathing and inhalation of essential oils.

According to some embodiments, nasal aromatic oil delivery system 100 comprises one or more exit channels 126. Exit channels 126 permit an air passageway so that when the reservoir is being filled at one or more fill ports 108, air may escape to outlets 128 from inlets 130 of reservoir 104 to permit easier refilling. In one embodiment, fill ports 108 are shaped to removably couple with a standard eyedropper. A standard eyedropper comprises a rubber bulb on one end connected to a small tube that ends with a pointed or rounded tip, which measures out liquid by drops. The pointed or rounded tip of a standard eyedropper typically comprises a surface that angles from the tube portion to the tip portion by an angle between 0 and 90 degrees, and typically between 0 and 60 degrees. This permits easy filling of reservoir 104 with standard essential oil vials, which typically contain a standard-sized eyedropper. In addition, or as an alternative, fill ports 108 comprise an angled surface, which mates with the outer angled surface of an eyedropper. For example, although eyedroppers comprise various shapes and sizes, embodiments contemplate fill ports 108 comprising a surface angled from approximately 0 to 60 degrees along the inner length of the accumulation chamber 112 such that any eyedropper would reversibly couple with fill ports 108. Although, a standard eyedropper is described, embodiments contemplate any eyedropper or other essential oil vials, according to particular needs.

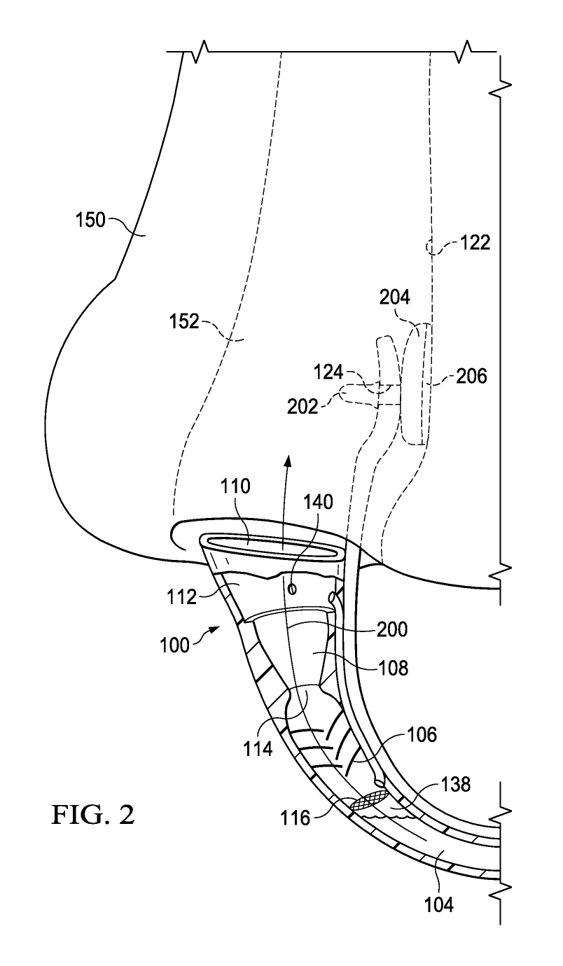

FIG. 2 illustrates nasal aromatic oil delivery system 100 of FIG. 1 in greater detail in accordance with the preferred embodiment. According to some embodiments, clips 118 may further comprise attachment holes 124. Attachment holes 124 each comprise a hole configured to receive peg 202 coupled with pad 204. Pad 204 is preferentially constructed of a soft material, such as latex, but may comprise any suitable material such as plastic, rubber, or the like. Pads 204 are sized to provide pad surfaces 206 that couple with inner surface 122 of nose 150 inside nostrils 152. Various-sized and/or various-shaped pads 202 may be provided with nasal aromatic oil delivery system 100 such that pads 202 are customizable and comfortably fit into nostrils 152 of various shapes and sizes. In one embodiment, pads 202 comprise two lobes with a restricted surface between the lobes, such that a first lobe is placed on a first side of clip 118, a second lobe is placed on a second side of clip 118, and the restricted surface is placed inside the attachment hole 124.

Nasal aromatic oil delivery system 100 provides delivery of any substance from reservoir 104 by vaporization, volatilization, and/or evaporation. According to a preferred embodiment, reservoir 104 is fillable with an odorant 120. Such odorants 120 may comprise one or more essential oils comprising therapeutic or pharmacological properties. The aromatic oil may comprise an essential oil such as, for example, oil of sweet orange, peppermint, cedarwood, lemon, eucalyptus, clove, spearmint, Litsea cubeba, Menthe arvensis, or the like. Additionally, embodiments of nasal aromatic oil delivery system 100 provide for odorant 120 to be placed in reservoir 104 for vaporization into nostril 152, which enhances strength, stamina, mental alertness, and endurance, while reducing fatigue. Furthermore, the design of the present nasal aromatic oil delivery system 100 comprises a single reservoir 104, which is fluidly coupled with both orifices 110 by a plurality of reservoir passages 138. Because nostrils are different sizes and pull different amounts of air from each orifice 110, the single reservoir design of the present invention ensures that single reservoir 104 provides vapor to both nostrils simultaneously, such that only one reservoir needs to be refilled—not a reservoir for each nostril individually.

In addition, or as an alternative, reservoir 104 may be filled with a substrate, such as a resin, which absorbs a liquid odorant 120 placed in reservoir 104. In this manner, spilling of odorant 120 is eliminated for at least the reason that odorant 120 will slowly evaporate from the substrate, while not being present in a spillable, liquid form. In some embodiments, odorant 120 is a liquid, solid, gel, or the like, comprising volatile odorant molecules, which are to be inhaled through nostril 152 of nose 150.

In some embodiments, the aroma from odorant 120 follows a pathway 200 from reservoir 104 into nostrils 152. Odorant 120 in reservoir 104, in a vapor or volatile form, flows from reservoir 104 along pathway 200 through reservoir passage 138 into restrictor channel 114, through accumulation chamber 112, out through orifice 110, and into nostril 152. In one embodiment pathway 200 comprises one or more of screen 116, louvers 106, and/or fill ports 108. Although only one passageway 200 is shown, embodiments contemplate pathways 200 for each of the left and right orifices 110.

Accumulation chambers 112 are configured to sit slightly below each nostril in order to not obstruct air intake, but still permit inhalation of vapor of odorant 120 with each breath. Accumulation chambers 112 provide a chamber whereby odorant 120 is concentrated before inhalation. Although FIG. 1 depicts an accumulation chamber 112 below each nostril 152, embodiments, contemplate nasal aromatic oil delivery system 100 having only a single accumulation chamber 112, according to particular needs.

Intake port 140 provides an opening through which ambient air can enter into the accumulation chamber. Intake port 140 is configured such that when orifice 110 sits directly underneath nostril 152, intake port 140 allows ambient air to enter into the accumulation chamber 112 which pushes the odorant into the nostril 152 when the pressure above the orifice 110 is decreased, such as with a breath. Ambient air, for purposes of this specification, means any gas, which is outside the device 100, such as, for example, air in the atmosphere.

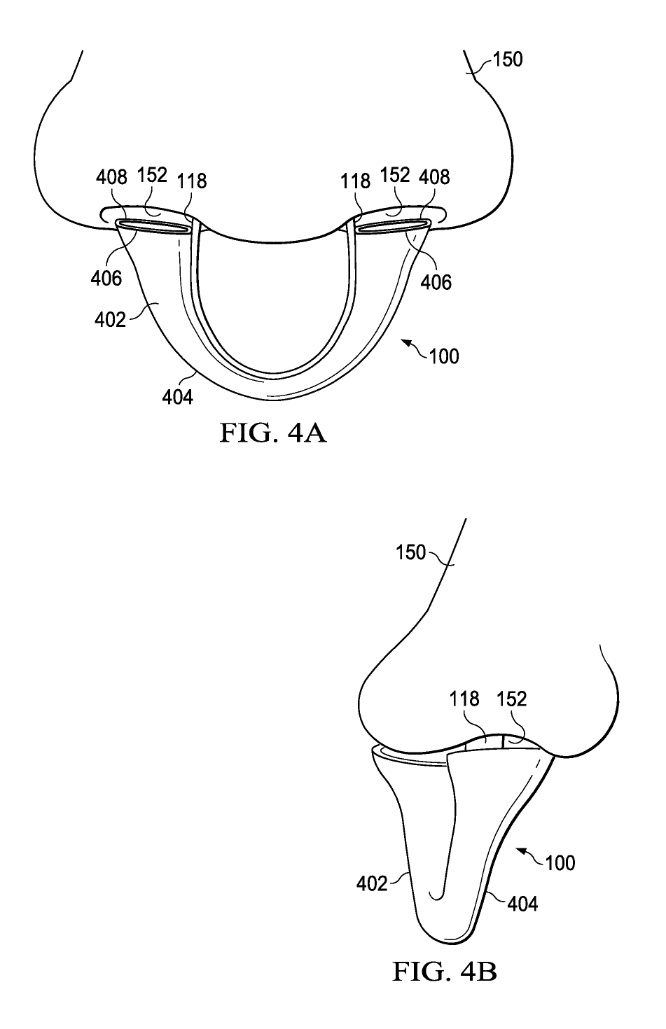

Orifice 110 comprises an opening from the accumulation chamber 112 into nostril 152. Orifice 110 comprises a shape suitable to sit below nostril 152 and to circumscribe an area of substantially the same shape as a nostril. As an example only and not by way of limitation, orifice 110 may comprise a circular, elliptical, or oval shape. According to some embodiments, orifice 110 is nostril-shaped, such as bean-shaped. According to some embodiments, orifice 110 comprises an oblong shape formed from joining one or more circles with one or more ellipses. According to some embodiments, a front surface of the perimeter of orifice 406 is higher than the back surface of the perimeter of orifice 408 (see FIGS. 4A and 4B) to permit nasal aromatic oil delivery system 100 to substantially contour to the three-dimensional shape of the surface surrounding the opening of the nostril 152.

Screens 116 provide a barrier to prevent foreign material other than odorant 120 from entering reservoir 104. According to some embodiments, screens 116 comprise an extremely fine mesh which, when coupled with the high surface tension and/or viscosity of oil, prevents oil from leaking out of nasal aromatic oil delivery system 100 when jostled or placed on its side.

Similarly, louvers 106 are constructed in a manner to permit odorant 120 to flow into reservoir 104 from fill port 108 and allow vapor or volatile odorant to flow into accumulation chamber 112, but prevent oil from leaking out of nasal aromatic oil delivery system 100. For example, according to some embodiments, louvers 106 comprise outward facing protrusions that form a helix along the inner surface of restrictor channel 114. The helix permits odorant to be trapped between the protrusions, with the helix facing downward such that odorant cannot move past the protrusions when the system is in sideways position, but also permits the odorant to drain back along the helix into reservoir 104 when the system is placed in an upright position.

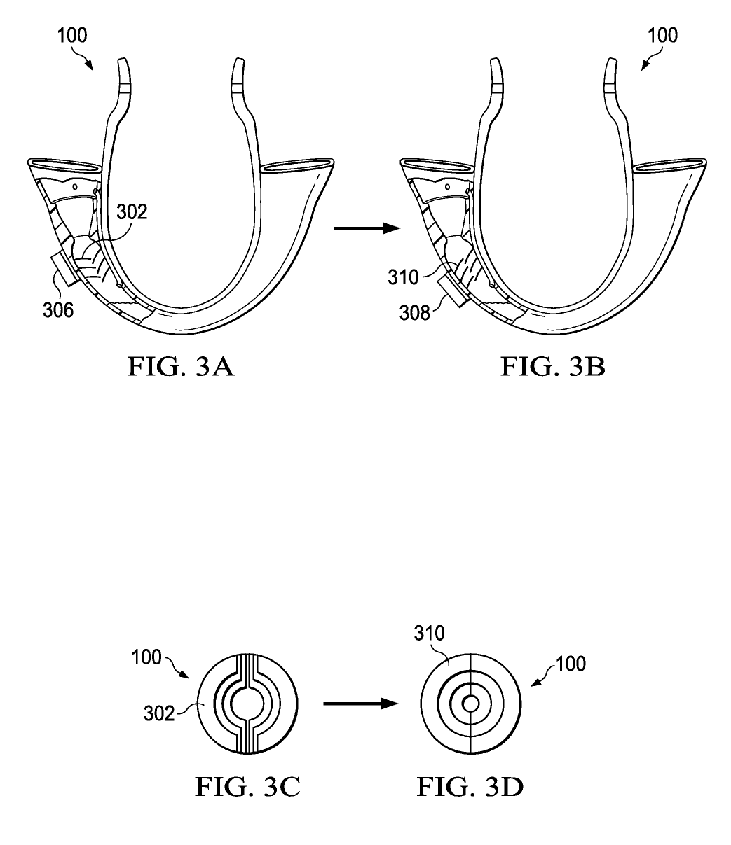

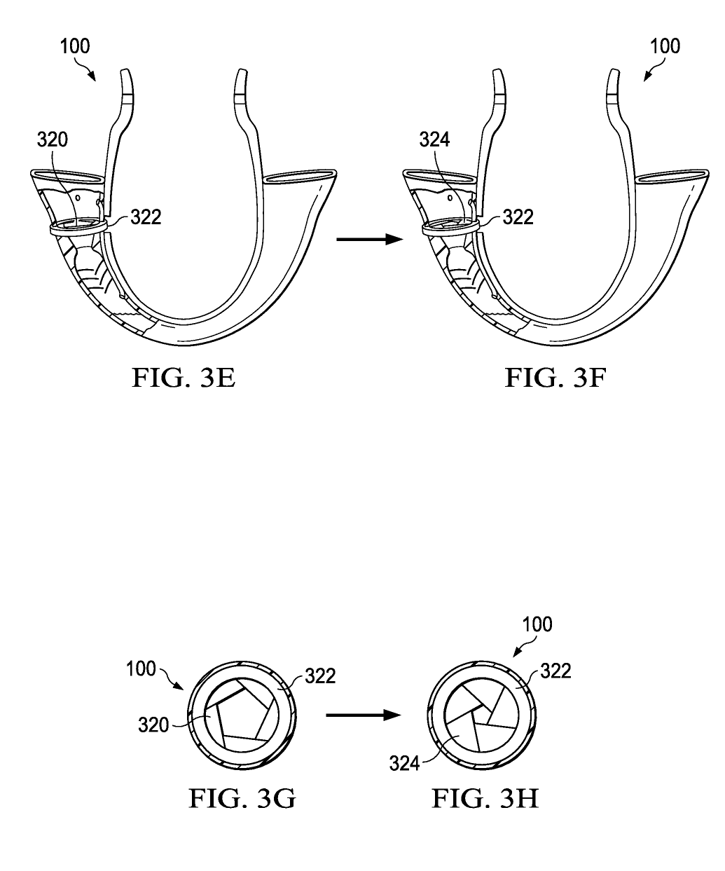

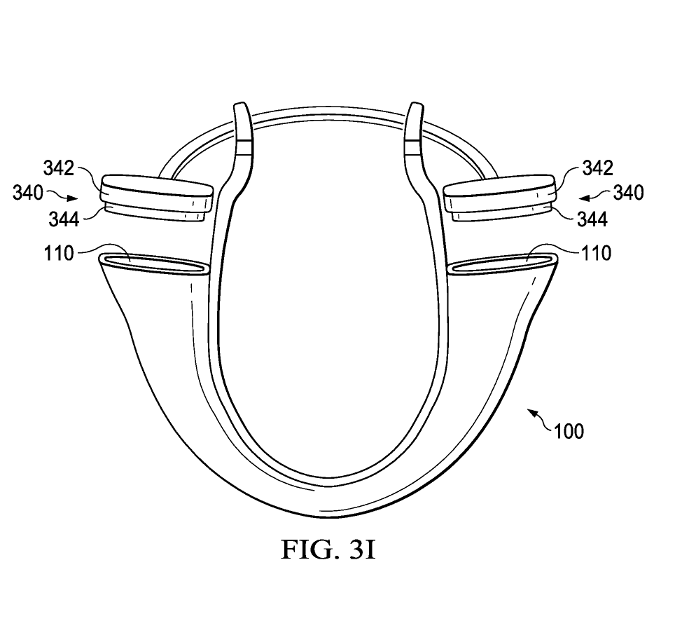

FIGS. 3C and 3D illustrate a top-down view of the louvers in a first position 302 and a second position 310. According to some embodiments, louvers 106 comprise one or more movable structures such as semicircles or an aperture, which is adjustable to increase, decrease, open or close the restrictor channel. For example, as shown in FIGS. 3A-3D, louvers 106 may be in a first position 302 when a switch is in a first position 306. Louvers 106 may be moved to a second position 310 when a switch is moved to a second position 308. Either the first or second positions of louvers 106 or switch may be a neutral, upward, or downward position according to particular needs. By way of example only and not by limitation, louvers 106 may be in a first position 102 sloping at a steep angle downward. This position permits reservoir 104 to be filled with an odorant 120 easily. Additionally or in the alternative, this position permits a greater amount of vapor to flow from reservoir 104 into nostril 152. Continuing with the example, when louvers 106 are moved to a second position 308, such as a less steeply inclined downward position, or a neutral position, odorant 120 may be prevented from passing through restrictor channel 114. This would permit nasal aromatic oil delivery system 100 to be stored on its side without odorant 120 from leaking out. Furthermore, this would permit the amount of vapor flowing through restrictor channel 114 and into nostril 152 to be lessened. In this manner, the amount of vapor released by nasal aromatic oil delivery system 100 is controllable.

FIGS. 3E-3H illustrate an aperture according to an embodiment, used to control the amount of vapor and/or odorant flowing through nasal aromatic oil delivery system 100. According to embodiments, rotation of a cuff 322 around the outer circumference of the aperture coupled to restrictor channel 114 increases or decreases the opening of the aperture. For example, an aperture may be in an open position 320 when the cuff is rotated in a first direction. The aperture may be in a closed position 324 when the cuff is rotated in a second direction. In this way, the amount of vapor passing through restrictor channel 114 can be precisely controlled, thereby controlling the amount of vapor entering into nostril 152. Similarly, when the aperture is in a completely closed position odorant 120 may be securely stored inside nasal aromatic oil delivery system 100 even when the system is on its side or upside down.

FIG. 3I illustrates a cap for nasal aromatic oil delivery system 100 according to embodiments. A cap 340 comprises an upper portion 342 and a lip 344. The lip 344 of cap 340 is configured to removably couple to orifice 110, such as by a protrusion on the lip 344 or the inner surface of orifice 110. In one embodiment, lip 344 snaps into an opposingly-positioned recess on the inner surface of orifice 110 or lip 344, respectively. Alternatively, both the lip 344 of cap 340 and the inner surface of orifice 110 may comprise protrusions which, when slid past each other, cause the cap to be removably coupled to the inner surface of orifice 110. Cap 340 prevents odorant 120 from spilling out of nasal aromatic oil delivery system 100 when nasal aromatic oil delivery system 100 is on its side or upside down. Additionally, cap 340 prevents odorant 120 from evaporating from nasal aromatic oil delivery system 100 when nasal aromatic oil delivery system 100 is not in use.

FIG. 4A illustrates a front view of nasal aromatic oil delivery system 100 coupled with nose 150 by clips 118 being inserted into nostrils 152. From a front view, nasal aromatic oil delivery system 100 comprises a forward-facing front surface 402 and a back or reverse surface 404. Also shown are a front surface of the perimeter of the orifice 406 and a back surface of the perimeter of the orifice 408 to permit the nasal aromatic oil delivery system 100 to substantially contour to the dimensions of nostril 152. According to some embodiments, the front surface of the perimeter of the orifice 406 is shaped to couple to the front surface of nostril 152, and the back surface of the perimeter of the orifice 408 is shaped to couple to the back surface of nostril 152, as discussed herein.

FIG. 4B illustrates a side view of nasal aromatic oil delivery system 100 coupled with nose 150 by clips 118 being inserted into nostrils 152. FIG. 4B illustrates nasal aromatic oil delivery system 100 comprising a front surface 402 and a back or reverse surface 404 coupled to nostril 152 by clips 118.

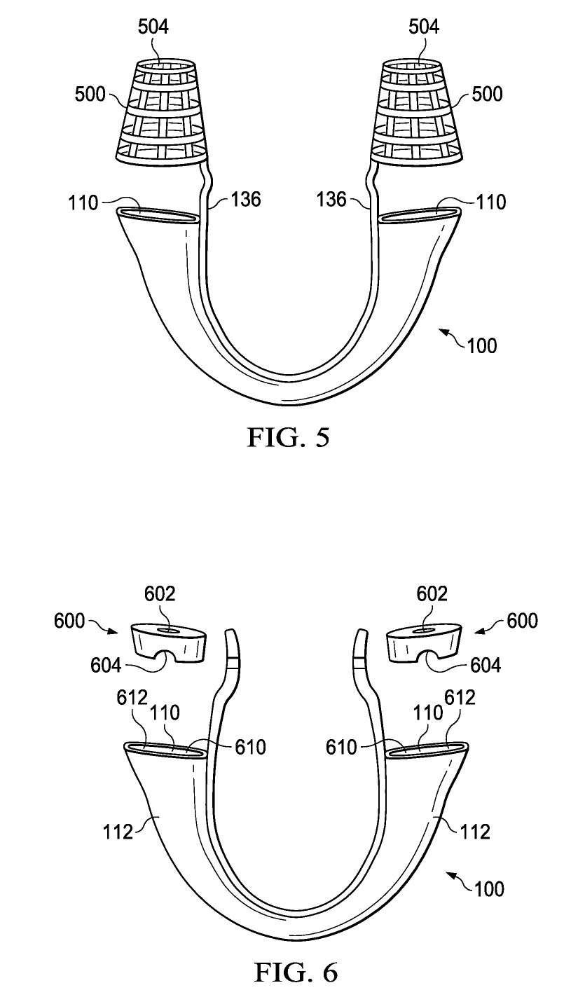

FIG. 5 illustrates an embodiment of a nasal aromatic oil delivery nasal aromatic oil delivery system 100 further comprising a nose clip 500. According to this embodiment, nose clip 500 comprises a basket shaped in the form of a truncated cone. The basket comprises interspaced elements in longitudinal and latitudinal orientations to permit air to flow from orifice 110 into a nostril through spaces 504 at the top of nose clip 500. A further feature of forming the basket of nose clip 500 in the shape of a truncated cone is that it dilates or expands the nostrils to provide increased airflow through a nose, permitting easier breathing and inhalation of essential oils. Nose clip 500 couples with nasal aromatic oil delivery system 100 via arms 136, which may comprise any length such that orifice 110 is held directly below the nostril.

FIG. 6 illustrates an embodiment of a nasal aromatic oil delivery system 100 comprising an interchangeable insert 600. According to some embodiments, interchangeable insert 600 comprises an insert intake hole 602 and an insert restriction channel 604. According to some embodiments, nasal aromatic oil delivery system 100 comprises an ambient air gap 610 and an odorant gap 612. Ambient air gap 610 comprises a hole from orifice 110 and accumulation chamber 112 to the ambient air through a side surface of nasal aromatic oil delivery system 100. Odorant gap 612 comprises a hole from accumulation chamber 112 through reservoir passage 138 to reservoir 104 (see FIG. 1). Both ambient air gap 610 and odorant gap 612 are preferably sized larger than insert intake hole 602 and insert restriction channel 604. When insert 600 is slid into accumulation chamber 112 the inset intake hole 602 couples to ambient air gap 610 and insert restriction channel 604 couples to odorant gap 612. This coupling of insert 600 to accumulation chamber 112 provides the ability to adjust the amount of odorant or ambient air entering into accumulation chamber 112 (and thus into a nostril) by providing inserts with various shaped insert intake holes 602 and insert restriction channels 604. By way of example only and not by way of limitation, embodiments contemplate providing nasal aromatic oil delivery system 100 with a variety of inserts 600 such that nasal aromatic oil delivery system 100 can be configured to provide various amounts of ambient air through insert intake holes 602 and various amounts of odorant through insert restriction channel 604. Such usefulness permits adjusting the amount of vapor to provide more or less vapor according to the appropriate dose, strength or sensitivity associated with the odorant (such an essential oil) providing the vapor. Similarly, adjusting the amount of ambient air flowing into the accumulation chamber permits greater airflow through nasal aromatic oil delivery system 100 to provide an appropriate amount according to the respiratory needs of a user.

According to some embodiments, inserts 600 comprise only an insert restriction channel 604. By way of example and not by limitation, inserts 600 may comprise a shape that couples to accumulation chamber 110 sits below the position of an ambient air gap 610 or intake port 140 thereby allowing various inserts 600 to be supplied that will alter the size of the restriction channel 114 but does not alter the size of the intake port 140 (see FIG. 1).

According to some embodiments, inserts 600 comprise no insert intake holes 602 and no insert restriction channels 602. An embodiment of an insert 600 with no holes provides a lid or closure such that odorant 120 does not flow out of leak from nasal aromatic oil delivery system 100 when insert 500 (see FIG. 5) is inserted into the accumulation chamber 112.

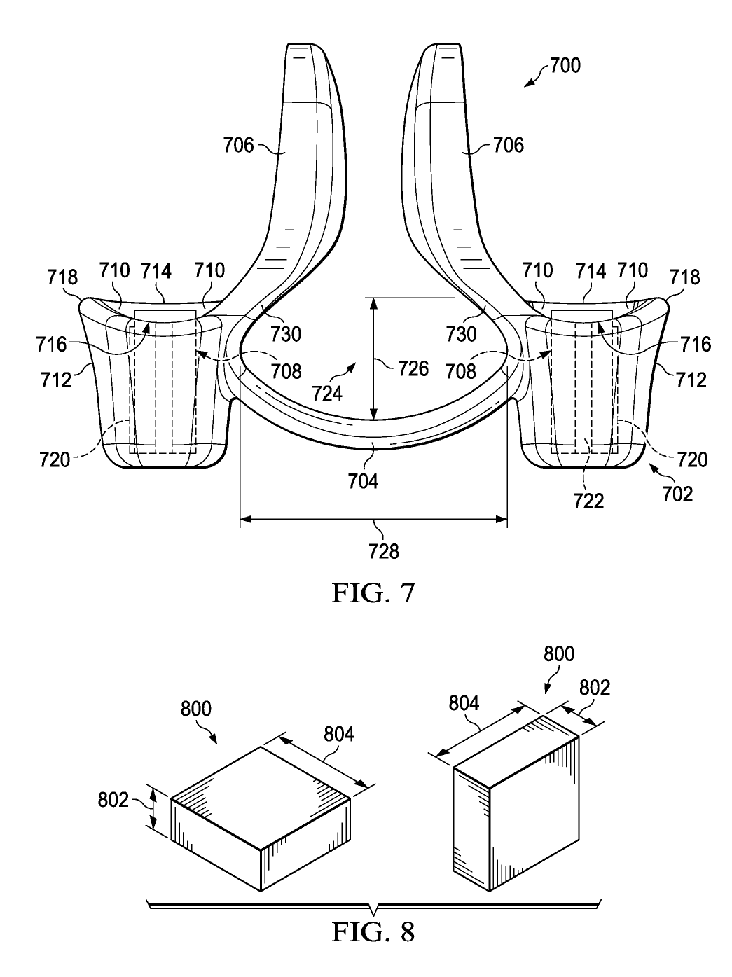

FIG. 7 illustrates a nasal aromatic oil delivery system 100 in accordance with a sport personal diffuser 700 embodiment. Although, sport personal diffuser 700 is described as comprising one or more various features, as described below, embodiments contemplate sport personal diffuser 700 comprising any one or more features described in connection with nasal aromatic oil delivery system 100. According to an embodiment, personal diffuser 700 may comprise a pair of wick cavity housings 702 coupled with a pillar 704 and a pair of internal nostril clips 706.

Internal nostril clips 706 permit sport personal diffuser 700 to be held firmly against the inside of a nose and to be properly positioned for maximum installation ease, comfort, and aroma vapor effectiveness. Internal nostril clips 706 may be coupled to wick cavity housings 702 by nostril clip tension regulators 730.

Nostril clip tension regulators 730 may comprise an internal ergonomically designed nostril clip tension regulator 730 that regulate holding pressure or tension of internal nostril clips 706 and may be designed to position wick cavity housing 702 for maximum essential oil aromatic vapor inhalation.

Pillar 704 may couple the pair of wick cavity housings 702. According to an embodiment, pillar 704 comprises an external nostril clip tension regulator and aerodynamic-enhanced design. Pillar 704 may add tension to the internal nostril clips 706 to hold sport personal diffuser 700 to inner nostril to position wick 800 (FIG. 8) in its pre-ergonomic-designed position for maximum essential oil vapor inhalation.

The pair of wick cavity housings 702 is designed to be aerodynamic and to hold a wick 800 inside wick cavities 708 situated substantially below a pair of nostrils.

FIG. 8 illustrates a pair of wicks 800 comprising a width 802 and height 804 and designed to be secured inside wick cavity 708. The width 802 and height 804 of wick 800 may be sized so as to fit within wick cavity 708. The wicks 800 may comprise an absorbent substrate or resin, such as POREX™ porous polyethylene substrate, that has been imbued or saturated with an odorant 120, such as essential oil or other vapor-releasing substance. The wick cavity 708 may comprise an internal cavity with a shape substantially similar to wick 800 or may be sized smaller or larger according to particular needs. Although the shape of wicks 800 and wick cavity 708 is described as having a particular shape, embodiments contemplate any shape and design of wicks 800 and wick cavities 708 according to particular needs.

According to an embodiment, the wicks 800 comprise a semi-rigid material that permits the wick 800 to deform slightly so that the wick may be sized slightly larger than the wick cavity 708 and the pressure from the wick 800 pushing against the walls of the cavity 708 secure the wick 800 to the inside of the wick cavity 708.

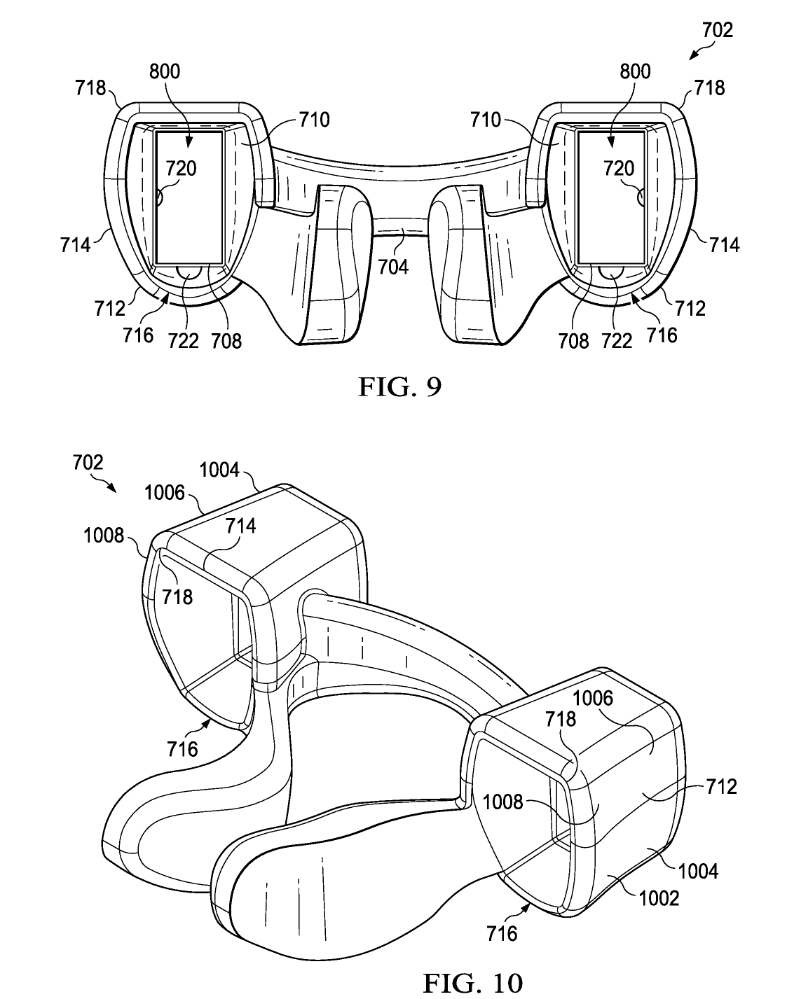

FIG. 9 illustrates a top down view of sport personal diffuser 700 according to an embodiment.

FIG. 9 illustrates ribs 720 and wick extraction channels 722 of sports personal diffuser 700. Wick cavities 708 may comprise ribs 720 and wick extraction channels 722. Rib 720 may comprise a tolerance-regulating rib that secures wick 800 inside wick cavity 708. The spatial tolerance of wicks 800 may comprise +/−0.012 inches. Rib 720 comprises a depth from the wall of the wick cavity 708 that is at least as great as the spatial tolerance of wick 800 such that the rib 720 holds the wick 800 securely inside the wick cavity 708 regardless of the precise wick 800 measurement. Although ribs 708 are indicated as being substantially at the side of the sport personal diffuser 700, embodiments contemplate any suitable number of ribs 708 or position along the interior of wick cavity 708 according to particular needs.

Wick extraction channels 722 of sport personal diffuser 700 may comprise a space or opening along the side of the wick cavity 708 that permits one or more of the following features: guiding a pick out tool for the removal or exchange of wick, cleaning and sterilizing sport personal diffusor 700, replacement of wick 800, and/or substitution of different types of odorants that are specific to the current situation. Although wick extraction channels 722 are indicated as being substantially at the front of the sport personal diffuser 700, embodiments contemplate any suitable position along the interior of wick cavity 708 according to particular needs.

Wick cavity housings 702 may further comprise enhanced vapor diffusion accumulation chambers 710. Enhanced vapor accumulation chambers 710 permit opening or exposing more of a top surface of wick 800 for accumulating and collecting more aromatic vapor for increased inhalation of vapor. Enhanced vapor accumulation chambers 710 may comprise a cavity defined by the top of the edges of the wick cavity housing 702 down to the top and sides of the wick 800 that are not covered by the wick cavity 708. Embodiments contemplate varying the sides of the wick cavity 708 to expose more or less of the wick 800 to the enhanced vapor accumulation chamber 710 to regulate the amount of diffusion of odorant 120 from wick 800. For example, a larger enhanced vapor accumulation chamber 710 permits more rapid diffusion, whereas a smaller enhanced vapor accumulation chamber 710 permits slower diffusion.

External portions of wick cavity housing 702 may comprise a directional force curve 712, an aroma vapor directional funnel 714, an induction offset curve 716, and a rear edge 718.

FIG. 10 illustrates a directional force curve 712 and an induction offset curve 716 of a sport personal diffuser 700, according to an embodiment.

Directional force curve 712 comprises an offset widening towards the back of sport personal diffuser 700 and permits, as sport personal diffusor 700 is in forward motion, reduced wind resistance, a stabilized position of wick cavity housing 702, and assistance in funneling aroma vapor up into nostrils, reducing vapor loss. According to some embodiments, directional force curve 712 comprises a surface that may be concave, convex, or a combination of both, that begins at the front 1002 and/or bottom 1004 of the wick cavity housing 702 and curves toward the back 1006 and/or top 1008 of wick cavity housing 702. For example, embodiments contemplate a concave directional force curve 712 that uses the airflow from the front 1002 of the wick cavity housing 702 toward the back 1006 of the wick cavity housing, caused by a user moving in a forward direction, as pressure to hold the personal diffusor 700 substantially in proper alignment. According to some embodiments, directional force curve 712 comprises a curve from the bottom 1004 of the wick cavity housing 702 toward the top 1008 of the wick cavity housing 702 configured to direct air upward toward the nose of a user of personal diffusor 700. According to some embodiments, directional force curve 712 comprises a combination of curves on the outer surface of wick cavity housing 702 to perform the functions just described.

Aroma vapor direction funnel 714 comprises sides and the back of the top 1008 of the outer surface of wick cavity housing 702 to funnel vapor upward into nasal passage.

Induction offset curve 716 permits adding an offset curve is an offset or dip in a front top curve of enhanced vapor diffusion accumulation chamber. According to an embodiment, induction offset curve 716 comprises an opening or recess on front 1002 surface of wick cavity housing 702 configured to permit increased airflow over wick 800.

Induction offset curve 716 may comprise an opening that dips below the top 1008 and front 1002 of wick cavity housing 702 or top surface of wick 800 so as to allow air induction or forcing of air as sport personal diffusor 700 is in a forward movement across wick 800 to enhance, forcing and always filling nostrils with aroma vapor even in between breaths.

Rear edge 718 may be particularly effective when used on a “sports” version of the system 700 and is slightly higher or raised than the side edges to corral and funnel upward as air from induction offset curve 716 wants to force vapor toward rear and over rear curve to upper lip losing aroma vapor to environment and losing its effectiveness. According to some embodiments, rear edge 718 comprises a gradual increase in the size of side edges toward the rear of wick cavity housing 702.

A columellar pocket 724 comprises a height 726 and a width 728 designed to accommodate the height and width of a nasal columella. The columellar pocket 724 may be ergonomically designed to comfortably accommodate a typical columella or space between nostrils.

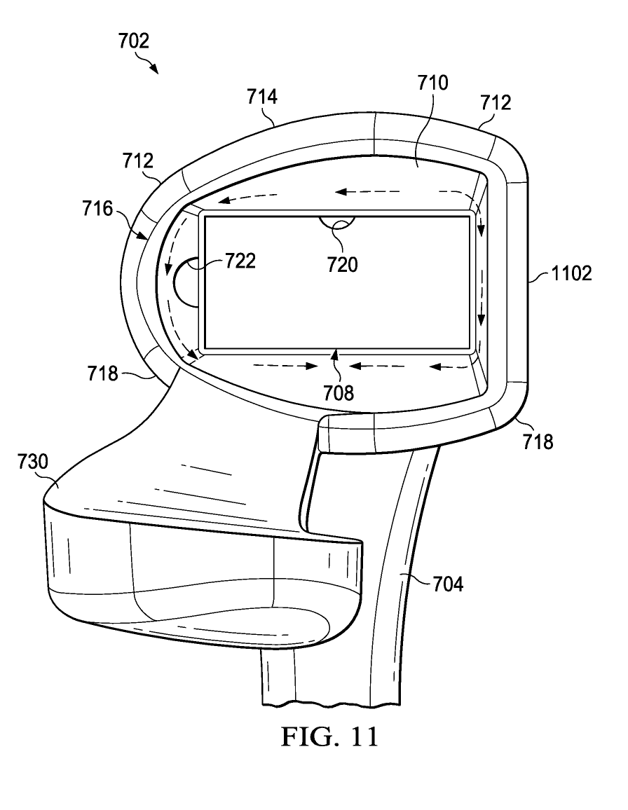

FIG. 11 illustrates the personal diffuser 700 of FIG. 7 in greater detail in accordance with an embodiment. Wick cavity housing 702 comprises ergonomically designed flat back 1102. Flat back 1102 comprises a back 1006 surface of wick cavity housing 708 that is substantially flat and squared off to permit user comfort, safety, and protection in case personal diffuser is position altered or dramatically forced into a user's upper lip area.

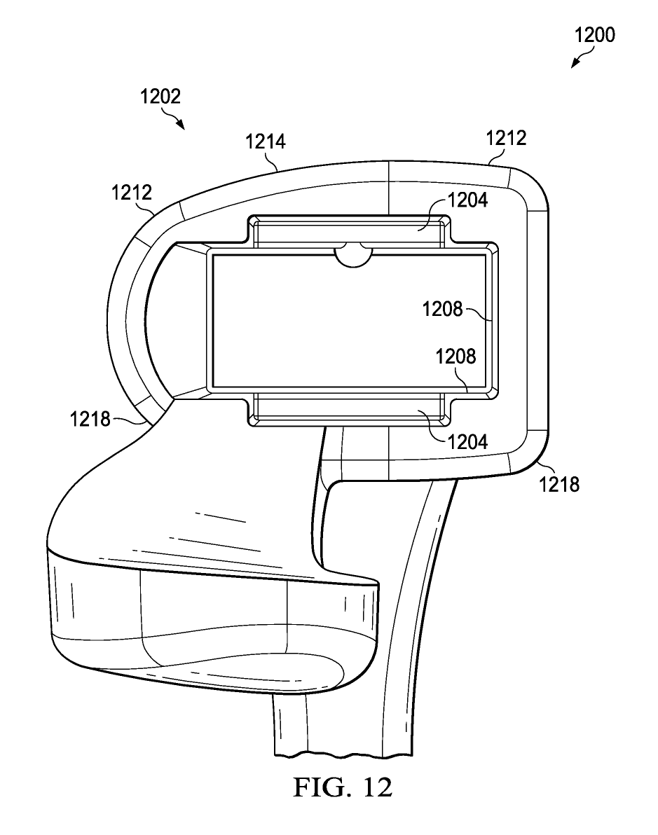

FIG. 12 illustrates a medical personal diffuser 1200 according to an embodiment. Medical personal diffuser 1200 comprises one or more features of sport personal diffuser 700 with differences, as described below.

Medical direction force curve 1212 of medical personal diffuser 1200 comprises straight sides from medical aroma vapor directional funnel 1214 down to base of medical wick cavity housing 1202. Medical personal diffuser 1200 may not comprise an induction offset curve 716. The top surface of wick cavity housing 1202 comprises uniform sides so as to resemble a round complete funnel matching the high point of rear edge 1218. Medical personal diffuser 1200 may not comprise aroma vapor direction funnel 714 or induction offset curve 716 permitting corralling and funneling aroma vapor up only as there will be no forward movement of medical personal diffuser 1200.

Medical personal diffuser 1200 may not comprise an enhanced vapor diffusion accumulation chamber 710. Instead, the walls of wick cavity 1208 will be flat against the sides of wick 800 all the way to the top of the wick 800. This permits diffusion from the top of wick 800 only, thereby permitting decreased vapor intensity and prolonging aroma vapor diffusion.

As medical personal diffuser 1200 may not comprise an enhanced vapor diffusion accumulation chamber 710, the walls of the wick cavity 1208 will cover the wick sides substantially completely in a medical personal diffuser 1200. According to an embodiment, a medical personal diffuser 1200 comprises two tweezer or tweezer-like tool slots 1204 on top sides of wick cavity that are ⅛ inch across and 1/16 inch deep in which a tweezer or tweezer-like tool can be inserted down the top sides of wick 800 for the purpose of extraction.

Embodiments also contemplate a youth personal diffuser that is a small-sized medical aromatic delivery system 1200 that has been decreased in overall size as to fit correctly and comfortably the smaller size of an adolescent nasal structure.

Reference in the foregoing specification to “one embodiment”, “an embodiment”, or “another embodiment” means that a particular feature, structure, or characteristic described in connection with the embodiment is included in at least one embodiment of the invention. The appearances of the phrase “in one embodiment” in various places in the specification are not necessarily all referring to the same embodiment.

While the exemplary embodiments have been shown and described, it will be understood that various changes and modifications to the foregoing embodiments may become apparent to those skilled in the art without departing from the spirit and scope of the present invention.

What is claimed is:

1. A personal diffuser, comprising:
a pair of wick cavity housings coupled with a pillar and a pair of internal nostril clips; and
each of the pair of wick cavity housings comprise a wick cavity configured to receive a wick.

2. The personal diffuser of claim 1, further comprising:
each of the pair of wick cavity housings further comprise an accumulation chamber.

3. The personal diffuser of claim 2, further comprising:
each of the pair of wick cavity housings further comprise a directional force curve with offset widening toward the back.

4. The personal diffuser of claim 3, further comprising:
each of the pair of wick cavity housings further comprise a tolerance regulating rib and wick extraction channel coupled to an interior surface of the wick cavity.

5. The personal diffuser of claim 4, further comprising:
each of the pair of wick cavity housings further comprise an aroma vapor directional funnel, an induction offset curve, and a rear edge configured to direct an aroma upward from the surface of the wick cavity.

6. The personal diffuser of claim 5, further comprising:
one or more wicks sized to fit into the wick cavity.

7. The personal diffuser of claim 6, wherein the one or more wicks are imbued with an odorant.

8. The personal diffuser of claim 7, further comprising:
each of the pair of wick cavity housings comprise a flat back surface.

9. A personal diffuser comprising:
a first wick cavity housing coupled with a second wick cavity housing by a pillar;
a first nostril clip coupled with the first wick cavity housing by a first nostril clip tension regulator;
a second nostril clip coupled with the second wick cavity housing by the second nostril clip tension regulator; and
each of the first wick cavity housing and the second wick cavity housing comprise an internal cavity.

10. The personal diffuser of claim 9, further comprising:
each of the first and second wick cavity housings comprise a base and straight sides from an aroma vapor directional funnel to the base.

11. The personal diffuser of claim 10, further comprising:
each of the first and second wick cavity housings comprise a rear edge and uniform sides whose height matches the height of a high point of the rear edge.

12. The personal diffuser of claim 11, wherein the internal cavity of the first and second wick cavity housings comprises flat sides.

13. The personal diffuser of claim 12, wherein the internal cavity of the first and second wick cavity housings comprises slots on at least one side.

14. The personal diffuser of claim 13, wherein the internal cavity of the first and second wick cavity housings comprises a tolerance regulating rib.

15. The personal diffuser of claim 14, further comprising:
one or more wicks sized to fit into the internal cavity of the first and second wick cavity housings.

16. The personal diffuser of claim 15, wherein the one or more wicks are imbued with an odorant.
